(19) United States
(12) Patent Application Publication
(10) Pub. No.: US 2022/0062512 A1
(43) Pub. Date: Mar. 3, 2022

(54) BLACK-COLORED MEDICAL DEVICE AND METHOD FOR PRODUCING SAME

(71) Applicant: MANI, INC., Utsunomiya-shi (JP)

(72) Inventors: Daigo Adachi, Utsunomiya-shi (JP); Kosuke Shinohara, Utsunomiya-shi (JP); Kazuhiko Matsutani, Utsunomiya-shi (JP)

(73) Assignee: MANI, INC., Utsunomiya-shi (JP)

(21) Appl. No.: 17/411,961

(22) Filed: Aug. 25, 2021

(30) Foreign Application Priority Data
Aug. 28, 2020 (JP) 2020-144489

(51) Int. Cl. A61L 31/14; A61B 17/06; A61L 31/08; C25D 7/00; C25D 5/18

(52) U.S. Cl. CPC A61L 31/14; A61B 17/06066; A61L 31/088; A61B 2017/06028; C25D 7/00; C25D 5/18; A61L 2420/02; A61B 17/06004

(57) ABSTRACT

An object of the present invention is to provide a method for producing a black-colored medical device being safe for living bodies with a shortened time for applying a pulse potential. The method for producing the black-colored medical device includes a step S1 of applying a square wave pulse potential to a stainless-steel medical device immersed in an electrolytic aqueous solution as one electrode for 40 to 90 minutes to form a colored passivation film on a surface of the medical device; and a step S3 of applying silicone to the medical device after applying the pulse potential. The method may include a step of curing a part of the medical device after the step of applying the pulse potential and before the step of applying silicone.

### TECHNICAL FIELD

The present invention relates to a black-colored medical device and a method for producing the medical device.

### BACKGROUND ART

Stainless-steel medical devices (e.g., knives and suture needles used in surgery) with black-dyed surfaces are sometimes used in order to avoid reduced visibility of treated area when light is reflected.

Immersion dyeing method for using sulfuric acid-chromic acid aqueous solution is known as a general metal blackening method. However, it is not suitable as a method for blackening medical devices because of the environmental impact of waste liquid treatment and the long coloring time. Even if other blackening methods are used, they must be safe for living bodies in order to apply the methods to medical devices, so methods in which heavy metals are used should be avoided.

A pulse electrolysis method, which has not been used in many medical devices, is recognized as safe for the living bodies, which can be used as a blacking method (see, for example, JPH02-107798A and JP2013-241664A). The pulse electrolysis method includes: immersing a metal such as stainless steel in an electrolytic solution such as a sulfuric acid solution; and applying a pulse potential to the metal to form a colored passivation film on the surface of the metal. This coloring method does not pose any particular danger to the living bodies, as long as no harmful metals are used, because the coloring is promoted by forming the film on the surface of the metal.

However, in the pulse electrolysis method, the color of the metal surface changes in accordance with the time of application of the pulse potential, and it generally takes about 180 minutes in order to change the color to black.

Therefore, it is difficult to solve the problem of long manufacturing time when medical devices are blacken using only the pulse electrolysis method.

### SUMMARY OF INVENTION

### Technical Problem

In view of this situation, an object of the present invention is to provide a method for producing a black-colored medical device being safe for living bodies with a shortened time for applying a pulse potential, and the black-colored medical device produced by such a method.

### Solution to Problem

A method for producing a black-colored medical device of the present invention includes: a step of applying a square wave pulse potential to a stainless-steel medical device immersed in an electrolytic aqueous solution as one electrode for 40 to 90 minutes to form a colored passivation film on a surface of the medical device (pulse electrolysis method); and a step of applying silicone to the medical device after applying the pulse potential.

The electrolytic solution may be a sulfuric acid solution of 3 mol/L or more and 5 mol/L or less. The pulse potential may have positive voltage of 1.2 V or more and 1.3 V or less, negative voltage of −0.6 V or more and −0.3 V or less, and frequency of 1 Hz or more and 5 Hz or less.

The method may include a step of curing a part of the medical device after the step of applying the pulse potential and before the step of applying silicone. Furthermore, the medical device may be a suture needle. The suture needle may have a blind hole having desired depth from an end sur face thereof. The part to be cured may be inner and outer surfaces of the blind hole.

The black-colored medical device may have a passivation film on the surface of the stainless-steel medical device. The passivation film does not contain environmentally regulated substances. The passivation film has an optical adjustment layer on a surface thereof.

The environmentally regulated substances that are not contained in the passivation film may be hexavalent chromium, chromic acid compounds, cyanide, or lead. The medical device may be a suture needle. The optical adjustment layer may be silicone.

### Advantageous Effect of the Invention

According to the present invention, it is possible to produce the black-colored medical device being safe for living bodies with a shortened production time. In addition, it is possible to achieve a black-colored surface of the medical device having an optical adjustment layer and a passivation film which does not contain the environmentally regulated substances, thus providing the black-colored medical device being safe for the living bodies.

### BRIEF DESCRIPTION OF THE DRAWINGS

FIG. 1 shows a flow chart for producing a black-colored medical device.

FIG. 2 shows an illustration of a pulse potential waveform.

FIG. 3 shows a perspective view of a suture needle.

### DESCRIPTION OF EMBODIMENTS

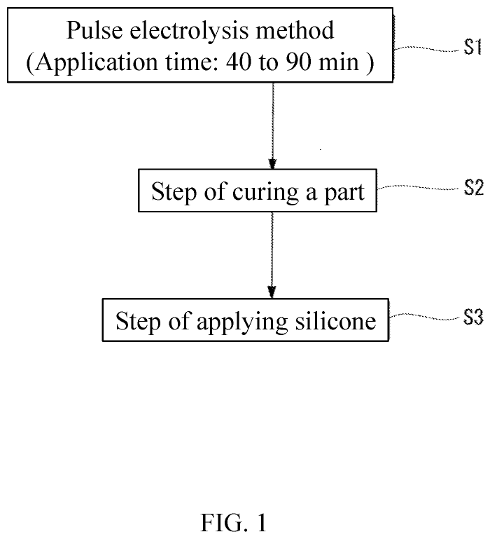

An embodiment of the present invention is described below with reference to the accompanying drawings. FIG. 1 shows a flow chart for producing a black-colored medical device.

An outline of a method for producing the black-colored medical device is as follows: the method includes a step S1 of applying a square wave pulse potential to a medical device immersed in an electrolytic aqueous solution to form a passivation film colored from dark brown to blue on a surface of the medical device (pulse electrolysis method); and a step S3 of applying silicone or the like to the medical device to form an optical adjustment layer after the step S1. The medical device includes stainless-steel devices including specifically knives, suture needles, and cannulae. In addition, the method may include a step S2 of curing a part of the medical device before applying silicone or the like.

The pulse electrolysis method Si includes a step of applying a square wave pulse potential to the stainless-steel medical device immersed in the electrolytic solution as one electrode for 40 to 90 minutes to form the colored passivation film on the surface of the medical device. The passivation film does not contain any environmentally regulated substances. Specifically, the passivation film does not contain the environmentally regulated substances such as hexavalent chromium, chromic acid compounds, cyanide and lead, and is safe for living bodies.

The aqueous electrolytic solution may be sulfuric acid solution, nitric acid solution, or phosphoric acid solution. If the sulfuric acid solution is used, the concentration is in the range of 3 mol/L to 5 mol/L, preferably 5 mol/L. The temperature of the aqueous electrolytic solution is in the range of 60° C. to 80° C., preferably 70° C.

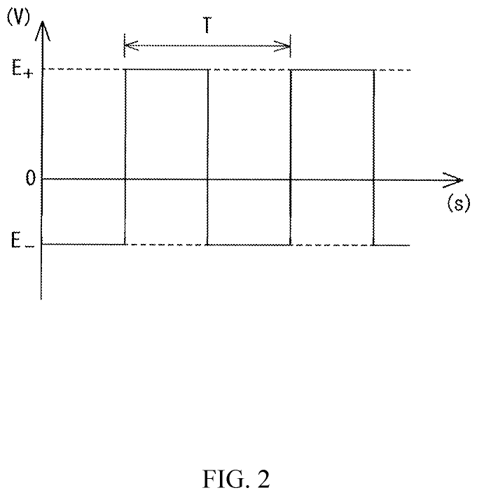

FIG. 2 shows an illustration of a pulse potential waveform. A horizontal axis shows application time of the pulse potential (seconds), and a vertical axis shows voltage (V) relative to a reference electrode. The waveform of the pulse potential is a square wave in which anode pulse voltage E+ and cathode pulse voltage E− relative to the reference electrode are applied alternately and repeatedly every predetermined time lapse. Pulse period 1/T of the pulse potential is within the ranget of 1 Hz to 5 Hz, prefebraly within the range of 2 Hz to 3 Hz. The anode pulse voltage E+ is wihtin the range of 1.2 V to 1.3 V, preferably 1.25 V, and the cathode pulse voltage E− is wihtin the range of −0.6 V to −0.3 V, preferably −0.5 V.

When the pulse electrolysis method is performed under these conditions, a colored passivation film is formed on the surface of the stainless-steel medical device, and the color of the passivation film changes according to the time of application of the pulse potential. Specifically, the color of the passivation film became gold when the time of application is 10 minutes, brown when the time of application is 20 minutes, dark brown when the time of application is 40 minutes, reddish purple when the time of application is 60 minutes, blue when the time of application is 90 minutes, yellow when the time of application is 120 minutes, and black when the time of application is 180 minutes. After that, the step S3 of applying silicone or the like to the medical device that has been colored by applying the pulsed potential is performed.

In the case of changing the color of the medical device to black, it is common practice to apply a pulse potential for 180 minutes to change the color of the medical device to black, and then apply silicone. In the present invention, the application of the pulse potential is terminated between 40 and 90 minutes, and then silicone or the like is applied to the medical device with the passive film colored from a dark brown to blue. Although silicone is colorless, the color of the medical device is changed to black because light is absorbed by the optical adjustment layer, such as silicone, formed on the surface of the colored passivation film.

The step of applying silicone may be a step of spraying liquid silicone on the surface of the medical device by a spray (see, for example, JP2012-50477A) or a step of immersing the medical device in a silicone solution, but not limited thereto. By coating the surface of the medical device with silicone, incision resistance can be reduced if the medical device is a knife, and piercing resistance can be reduced if the medical device is a suture needle. In other words, any materials other than silicone may be used to form the optical adjustment layer, but silicone is the most suitable material considering its feature of reducing the resistance.

After the step S2 of curing a part of the medical device after applying a pulse potential, the step S3 of applying silicone or the like may be performed. In other words, the cured part has a different color (e.g., blue) from the black color because silicone is not applied to the part. Thus, it is possible to distinguish the silicone coated part from the uncoated part. Therefore, it becomes possible to judge whether silicone is properly coated or not, and furthermore, it becomes easy to judge whether silicone is peeling off or not when the medical device is used, for example, when incision is continuously performed with a knife or when insertion is continulusly performed with a suture needle.

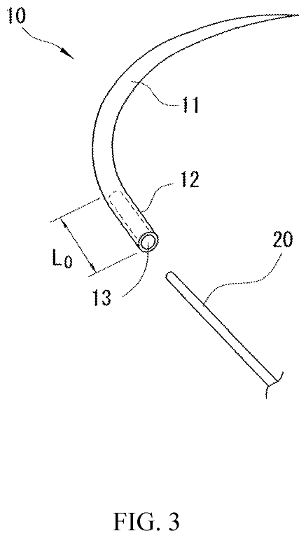

When the medical device is a suture needle capable of attaching a suture thread to a blind hole thereof, it is better to cure inner and outer surfaces of the blind hole. FIG. 3 shows a perspective view of the suture needle. In the suture needle 10, the suture thread 20 is inserted into the blind hole 13 and then the suture needle 10 is swaged to attach the suture thread 20. In other words, if the inner surface of the blind hole 13 is coated with silicone, the suture thread 20 will easily come loose. Also, if silicone is applied to the outer surface of the blind hole 13, it will adhere to a die used for swaging, which will require wiping off. Therefore, a swaged part 12 corresponding to the depth L₀ of the blind hole 13 from an end surface of the suture needle 10 is preferably cured to prevent silicone from being applied.

### DESCRIPTION OF THE REFERENCE NUMERAL

10 Suture needle
11 Silicone-coating area
13 Swaged part
12 Blind hole
20 Suture thread

What is claimed is:

1. A method for producing a black-colored medical device comprising:
applying a square wave pulse potential to a stainless-steel medical device immersed in an electrolytic aqueous solution as one electrode for 40 to 90 minutes to form a colored passivation film on a surface of the medical device; and applying silicone to the medical device after applying the pulse potential.

2. The method according to claim 1, wherein the electrolytic solution is a sulfuric acid solution of 3 mol/L or more and 5 mol/L or more, and
the pulse potential has positive voltage of 1.2 V or more and 1.3 V or less, negative voltage of −0.6 V or more to −0.3 V or less, and frequency of 1 Hz or more and 5 Hz or less.

3. The method according to claim 2, the method further comprises curing a part of the medical device after applying the pulse potential and before applying silicone.

4. The method according to claim 3, wherein the medical device is a suture needle,
the suture needle has a blind hole having desired depth from an end surface thereof, and
the part to be cured may be inner and outer surfaces of the blind hole.

5. A black-colored medical device, comprising a passivation film on the surface of a stainless-steel medical device, wherein
the passivation film does not contain environmentally regulated substances, and
the passivation film has an optical adjustment layer on a surface thereof.

6. The black-colored medical device according to claim 5, wherein the environmentally regulated substances that are not contained in the passivation film is hexavalent chromium, chromic acid compounds, cyanide, or lead.

7. The black-colored medical device according to claim 5, wherein the medical device is a suture needle, and
the optical adjustment layer is silicone.
